(19) United States
(12) Patent Application Publication
(10) Pub. No.: US 2013/0006092 A1
(43) Pub. Date: Jan. 3, 2013

(54) Magnetic Nanoparticle Compositions and Methods of Use Thereof

(75) Inventors: Richard Ferrans, Chicago, IL (US); Mridula Pottathil Sherin, Satellite Beach, FL (US); Raveendran Pottathil, La Jolla, CA (US)

(73) Assignee: NANOVORTEX LLC, Chicago, IL (US)

(21) Appl. No.: 13/525,324

(22) Filed: Jun. 17, 2012

Related U.S. Application Data
(60) Provisional application No. 61/498,519, filed on Jun. 17, 2011.

(51) Int. Cl. A61B 5/055; A61N 2/10; C07K 16/00; C07H 21/00; B82Y 5/00

(52) U.S. Cl. 600/411; 530/391.3; 536/23.1; 600/12; 977/773; 977/915; 977/927

(57) ABSTRACT

The present invention relates to diagnosis and treatment of medical conditions using magnetic nanoparticle compositions.

The present application claims priority to U.S. provisional application 61/498,519 filed Jun. 17, 2011.

### FIELD OF INVENTION

The present invention relates to diagnosis and treatment of medical conditions using magnetic nanoparticle compositions.

### BACKGROUND

Nanomedicine is the medical application of nanotechnology. Nanomedicine seeks to deliver a valuable set of research tools and clinically useful devices. Nanomedical approaches to drug delivery center on developing nanoscale particles or molecules to improve drug bioavailability. Bioavailability refers to the presence of drug molecules where they are needed in the body and where they will do the most good. Drug delivery focuses on maximizing bioavailability both at specific places in the body and over a period of time. This can potentially be achieved by molecular targeting by nanoengineered devices. It is all about targeting the molecules and delivering drugs with cell precision. The success of particular therapeutics hinges on targeting the molecules and delivering therapeutics with cell precision.

In vivo imaging is another area where tools and devices are being developed. Using nanoparticle contrast agents, images such as ultrasound and MRI have a favorable distribution and improved contrast. The new methods of nanoengineered materials that are being developed might be effective in treating illnesses and diseases such as cancer.

Protein and peptides exert multiple biological actions in human body and they have been identified as showing great promise for treatment of various diseases and disorders. These macromolecules are called biopharmaceuticals. Targeted and/or controlled delivery of these biopharmaceuticals using nanomaterials like nanoparticles is an emerging field called nanobiopharmaceutics, and these products are called nanobiopharmaceuticals.

The small size of nanoparticles endows them with properties that can be very useful in oncology, particularly in imaging. Another nanoproperty, high surface area to volume ratio, allows many functional groups to be attached to a nanoparticle, which can seek out and bind to certain tumor cells. Additionally, the small size of nanoparticles (10 to 100 nanometers), allows them to preferentially accumulate at tumor sites (because tumors lack an effective lymphatic drainage system).

To date, the field of nanomedicine has been focused on improving bioavailability of existing therapeutics by nanosizing them or enclosing them within an nanoparticle capable of delivering the therapuetic to the appropriate site.

There are recent advancements in nanomedicine where the particle itself acts as both the targeting and therapeutic agent.

Magnetic nano-particles are a particular type of nano-particle. Under the influence of a magnet, these particles spin and the heat generated is believed to cause cell damage, thereby acting as a therapeutic agent.

### SUMMARY OF THE INVENTION

The present invention provides a nano-magnetic composition capable of polyvalent coordinated binding to target molecules and under the influence of a magnet, causes cell damage without the generation of heat or physical tearing. The magnetic nanoparticle is capable of being imaged and is also the therapeutic agent. Specificity is derived from the functionalization of the nanoparticle with target specific ligands.

The present invention provides methods of diagnosing and treating a variety of medical conditions comprising administering a nano-composition to a mammal, applying a weak magnet to change the magnetic spin of the composition thereby inducing a non-heating mechanism of action wherein ion channels of target cells are disrupted and the targeted cells die. This feature differentiates the present invention from traditional magnetic nanoparticles. In addition, the present invention addresses the issue of heterogenous cell populations and treatment resistance by administering a plurality of nano-particles with different targeting ligands attached to it—thereby targeting the variety of cell types within a given cell group.

### DETAILED DESCRIPTION OF THE INVENTION

### Nanoparticles

Nanoparticles are particles that have one dimension that is 100 nanometers or less in size. The properties of many conventional materials change when formed from nanoparticles. This is typically because nanoparticles have a greater surface area per weight than larger particles; this causes them to be more reactive to certain other molecules.

Particles are further classified according to size: in terms of diameter, fine particles cover a range between 100 and 2500 nanometers. On the other hand, ultrafine particles are sized between 1 and 100 nanometers. Similar to ultrafine particles, nanoparticles are sized between 1 and 100 nanometers. Nanoparticles may or may not exhibit size-related properties that differ significantly from those observed in fine particles or bulk materials. Although the size of most molecules would fit into the above outline, individual molecules are usually not referred to as nanoparticles.

Nanoclusters have at least one dimension between 1 and 10 nanometers and a narrow size distribution. Nanopowders are agglomerates of ultrafine particles, nanoparticles, or nanoclusters. Nanometer-sized single crystals, or single-domain ultrafine particles, are often referred to as nanocrystals.

Nanoparticles can be made from metals, non-metals, polymers of aminoacids, and sugars just to name a few.

Nanoparticles are of great scientific interest as they are effectively a bridge between bulk materials and atomic or molecular structures. A bulk material should have constant physical properties regardless of its size, but at the nano-scale this is often not the case. Size-dependent properties are observed such as quantum confinement in semiconductor particles, surface plasmon resonance in some metal particles and superparamagnetism in magnetic materials. The properties of materials change as their size approaches the nanoscale and as the percentage of atoms at the surface of a material becomes significant.

The interesting and sometimes unexpected properties of nanoparticles are partly due to the aspects of the surface of the material dominating the properties in lieu of the bulk properties. Nanoparticles exhibit a number of special properties relative to bulk material. For example, the bending of bulk copper (wire, ribbon, etc.) occurs with movement of copper atoms/clusters at about the 50 nm scale. Copper nanoparticles smaller than 50 nm are considered super hard materials that do not exhibit the same malleability and ductility as bulk copper.

Extensive libraries of nanoparticles, composed of an assortment of different sizes, shapes, and materials, and with various chemical and surface properties, have already been constructed. The field of nanotechnology is under constant and rapid growth and new additions continue to supplement these libraries.

### Synthesis

There are several methods for creating nanoparticles, including both attrition and pyrolysis. In attrition, macro or micro scale particles are ground in a ball mill, a planetary ball mill, or other size reducing mechanism. The resulting particles are air classified to recover nanoparticles. In pyrolysis, a vaporous precursor (liquid or gas) is forced through an orifice at high pressure and burned. The resulting solid (a version of soot) is air classified to recover oxide particles from by-product gases. Pyrolysis often results in aggregates and agglomerates rather than single primary particles.

Thermal plasma can also deliver the energy necessary to cause evaporation of small micrometer size particles. The thermal plasma temperatures are in the order of 10,000 K, so that solid powder easily evaporates. Nanoparticles are formed upon cooling while exiting the plasma region.

The main types of the thermal plasma torches used to produce nanoparticles are dc plasma jet, dc arc plasma and radio frequency (RF) induction plasmas. In the arc plasma reactors, the energy necessary for evaporation and reaction is provided by an electric arc which is formed between the anode and the cathode. For example, silica sand can be vaporized with an arc plasma at atmospheric pressure. The resulting mixture of plasma gas and silica vapour can be rapidly cooled by quenching with oxygen, thus ensuring the quality of the fumed silica produced. In RF induction plasma torches, energy coupling to the plasma is accomplished through the electromagnetic field generated by the induction coil. The plasma gas does not come in contact with electrodes, thus eliminating possible sources of contamination and allowing the operation of such plasma torches with a wide range of gases including inert, reducing, oxidizing and other corrosive atmospheres.

The working frequency is typically between 200 kHz and 40 MHz. Laboratory units run at power levels in the order of 30-50 kW while the large scale industrial units have been tested at power levels up to 1 MW. As the residence time of the injected feed droplets in the plasma is very short it is important that the droplet sizes are small enough in order to obtain complete evaporation. The RF plasma method has been used to synthesize different nanoparticle materials, for example synthesis of various ceramic nanoparticles such as oxides, carbours/carbides and nitrides of Ti and Si.

Inert-gas condensation is frequently used to make nanoparticles from metals with low melting points. The metal is vaporized in a vacuum chamber and then supercooled with an inert gas stream. The supercooled metal vapor condenses into nanometer-sized particles, which can be entrained in the inert gas stream and deposited on a substrate or studied in situ.

### Sol-Gel

The sol-gel process is a wet-chemical technique (also known as chemical solution deposition) widely used recently in the fields of materials science and ceramic engineering. Such methods are used primarily for the fabrication of materials (typically a metal oxide) starting from a chemical solution (sol, short for solution) which acts as the precursor for an integrated network (or gel) of either discrete particles or network polymers.

Typical precursors are metal alkoxides and metal chlorides, which undergo hydrolysis and poly-condensation reactions to form either a network “elastic solid” or a colloidal suspension (or dispersion)—a system composed of discrete (often amorphous) submicrometer particles dispersed to various degrees in a host fluid. Formation of a metal oxide involves connecting the metal centers with oxo (M-O-M) or hydroxo (M-OH-M) bridges, therefore generating metal-oxo or metal-hydroxo polymers in solution. Thus, the sol evolves towards the formation of a gel-like diphasic system containing both a liquid phase and solid phase whose morphologies range from discrete particles to continuous polymer networks.

In the case of the colloid, the volume fraction of particles (or particle density) may be so low that a significant amount of fluid may need to be removed initially for the gel-like properties to be recognized. This can be accomplished in any number of ways. The simplest method is to allow time for sedimentation to occur, and then pour off the remaining liquid. Centrifugation can also be used to accelerate the process of phase separation.

Removal of the remaining liquid (solvent) phase requires a drying process, which is typically accompanied by a significant amount of shrinkage and densification. The rate at which the solvent can be removed is ultimately determined by the distribution of porosity in the gel. The ultimate microstructure of the final component will clearly be strongly influenced by changes implemented during this phase of processing. Afterwards, a thermal treatment, or firing process, is often necessary in order to favor further poly-condensation and enhance mechanical properties and structural stability via final sintering, densification and grain growth. One of the distinct advantages of using this methodology as opposed to the more traditional processing techniques is that densification is often achieved at a much lower temperature.

The precursor sol can be either deposited on a substrate to form a film (e.g. by dip-coating or spin-coating), cast into a suitable container with the desired shape, or used to synthesize powders (e.g. microspheres, nanospheres). The sol-gel approach is a cheap and low-temperature technique that allows for the fine control of the product's chemical composition. Even small quantities of dopants, such as organic dyes and rare earth metals, can be introduced in the sol and end up uniformly dispersed in the final product. It can be used in ceramics processing and manufacturing as an investment casting material, or as a means of producing very thin films of metal oxides for various purposes. Sol-gel derived materials have diverse applications in optics, electronics, energy, space, biosensor, medicine (e.g. controlled drug release) and separation (e.g. chromatography) technology

### Ligands

The nanoparticles can be directed to relevant sites by the addition of particular ligands. These ligands enable targeting of the nanoparticles to the site requiring imaging and/or treatment. Ligands can include but are not limited to antibodies and nucleic acid aptamers. Appropriate antibodies would be those that, for instance, bind to molecules preferentially expressed on cancer cells or bacterial/viral infected cells. Appropriate nucleic acid aptamers would be those that, for instance, have a conformational structure that enabled preferential binding to particular pathogens, cancer cells, tumor cells, cells infected with a pathogen, or particular tissue types.

The surface coating of nanoparticles is crucial to determining their properties. In particular, the surface coating can regulate stability, solubility and targeting. A coating that is multivalent or polymeric confers high stability. For biological applications, the surface coating should be polar to give high aqueous solubility and prevent nanoparticle aggregation. In serum or on the cell surface, highly charged coatings promote non-specific binding, while polyethylene glycol linked to terminal hydroxyl or methoxy groups repel non-specific interactions.

Nanoparticles can be linked to biological molecules which can act as address tags, to direct the nanoparticles to specific sites within the body specific organelles within the cell, or to follow specifically the movement of individual protein or RNA molecules in living cells. Common address tags are monoclonal antibodies, aptamers, streptavidin or peptides. These targeting agents should ideally be covalently linked to the nanoparticle and should be present in a controlled number per nanoparticle. Multivalent nanoparticles, bearing multiple targeting groups, can cluster receptors, which can activate cellular signaling pathways, and give stronger anchoring.

Ligands include but are not limited to antigens, antibodies, nucleic acids, and aptamers.

Targets include but are not limited to bacteria, viruses, fat cells, tumor cells, and arterial plaque cells.

### Polyvalent Binding

In biochemistry, a macromolecule exhibits cooperative binding if its affinity for its ligand changes with the amount of ligand already bound. Cooperative binding is a special case of allostery. Cooperative binding requires that the macromolecule have more than one binding site, since cooperativity results from the interactions between binding sites. If the binding of ligand at one site increases the affinity for ligand at another site, the macromolecule exhibits positive cooperativity.

In binding of a ligand to a target molecule, mutually-matching heteropolyvalency yields enhanced ‘selectivity’, or ‘discriminatory power’, as is observed in both natural and artificial systems.

In polyvalent binding, molecular flexibility of ligand or target may either favor affinity and discrimination (minimizing the enthalpy lost due to strain on binding) or be adverse (through loss of conformational entropy upon binding at sites after the first).

The binding of appropriate polyvalent ligands to polyvalent targets yields increased affinity and discrimination as compared with monovalent binding. This may be more readily appreciated and possibly also of greater technical importance when the interaction is heteropolyvalent, and the enhanced discrimination should be thought of as a phenomenon distinct from the more obviously expected increase in strength of binding.

### Targets

Medical targets for which the present invention would be useful include but are not limited to solid tumor cancers, fat ablation, bacterial and viral infections, and arterial plaques. More specifically, markers include but are not limited to the following:

B-cell Surface Markers: Activated B-cells: CD28, CD38, CD69, CD80, CD83, CD86, DPP4, FCER2, IL2RA, TNFRSF8, CD70 (TNFSF7). Mature B-cells: CD19, CD22, CD24, CD37, CD40, CD72, CD74, CD79A, CD79B, CR2, IL1R2, ITGA2, ITGA3, MS4A1, ST6GAL1. Other B-cell Surface Markers: CD1C, CHST10, HLA-A, HLA-DRA, NT5E.

T-cell surface markers: Cytotoxic T-cells: CD8A, CD8B1.Helper T-cells: CD4. Activated T-cells: ALCAM, CD2, CD38, CD40LG, CD69, CD83, CD96, CTLA4, DPP4, HLA-DRA, IL12RB1, IL2RA, ITGA1, TNFRSF4, TNFRSF8, CD70 (TNFSF7). Other T-cell Surface Markers: CD160, CD28, CD37, CD3D, CD3G, CD3Z, CD5, CD6, CD7, FAS, KLRB1, KLRD1, NT5E, ST6GAL1.

Natural Killer (NK) Cell Surface Markers: CD2, CD244, CD3Z, CD7, CD96, CHST10, IL12RB1, KLRB1, KLRC1, KLRD1, NCAM1.

Monocyte and Macrophage Cell Surface Markers: Activated Macrophages: CD69, ENG, FCER2, IL2RA. Other Monocyte and Macrophage Surface Markers: C5R1, CD163, CD40, CD63, CD74, CD86, CHST10, CSF1R, DPP4, FCGR1A, HLA-DRA, ICAM2, IL1R2, ITGA1, ITGA2, S100A8, TNFRSF8, CD70 (TNFSF7).

Endothelial Cell Surface Markers: ENG, ICAM2, NOS3, PECAM1, SELP, VCAM1, VWF.

Smooth Muscle Cell Surface Markers: MYH10, MYH9, MYOCD.

Dendritic Cell Surface Markers: CD1A, CD209, CD40, CD83, CD86, CR2, FCER2.

Mast Cell Surface Markers: C5R1, FCER1A, FCER2, TPSAB1.

Fibroblast (Stromal) Surface Markers: ALCAM, COL1A1, COL1A2.

Epithelial Cell Surface Markers: CD1D, KRT18, KRT5, KRT8, TACSTD1.

Adipocyte Surface Markers: RETN

Cancer markers include but are not limited to CD44v [several types of cancer, metastasis marker], MCA [breast], CEA [colon and breast], CA19-9 [pancreatic], HER2/neu [breast cancer], CA-15-3 [breast cancer], CA-125 [ovarian], AFP [hepatocellular carcinoma], and PSA [prostate].

Specific cell markers for any variety of tissues and physiological systems are well known in the art Such markers can be used with the magnetic nanoparticles of the present invention.

### Combined Diagnosis and Therapeutics

Nanoparticles form a convenient platform for combined diagnostics and therapuetics. Each particle can carry highly specific targeting ligands, treatment moieties, different imaging probes, and various combinations thereof. To date, the imaging probes contemplated have been attached to the particle. The present invention proposes that the magentic nanoparticle itself serves as the imaging probe and therapeutic moiety while the specific targeting ligands enable delivery of the nanoparticle to target cells of interest. Alternatively, the magnetic nanoparticle is capable of having an imaging probe attached to it to be detected by corresponding imaging systems.

Nanoparticle-based “molecular” imaging represents advancement in diagnostic imaging. Inorganic nanoparticles (NPs) including semiconductor quantum dots (QDs), iron oxide NPs and gold NPs have been developed as contrast agents for diagnostics by molecular imaging. Compared with traditional contrast agents, NPs offer several advantages: their optical and magnetic properties can be tailored by engineering the composition, structure, size and shape; their surfaces can be modified with ligands to target specific biomarkers of disease; the contrast enhancement provided can be equivalent to millions of molecular counterparts; and they can be integrated with a combination of different functions for multimodal imaging.

The present invention comprises magnetic nanoparticles functionalized with polyvalent ligands capable of binding the same target cells and wherein, upon subjection to a magnetic field, the nanoparticle vortices shift and this shift is detected using magnetic resonance imaging techniques. The present invention will also use a variety of techniques to temporarily and/or reversibly bind paramagnetic particles to targets to achieve imaging through magnetic resonance imaging or though use of contrast agents that will enhance binding or resolution of binding targets.

Alternatively, the magnetic nanoparticle could be functionalized with a traditional imaging fluorophore as well as a targeting ligand. This would enable imaging through methods known in the art such as but not limited to PET scan, contrast imaging, fusion imaging, nuclear medical imaging, and other imaging techniques.

The present invention comprises magnetic nanoparticles capable of being functionalized via a combined medical imaging and therapeutic device such as those utilized in fluoroscopic interventional radiology suites, except that this device would rely on magnetic imaging to identify targets, followed by specific magnetic frequencies, amplitudes, lengths of exposure, field strengths in specific sequences to achieve a therapeutic effect. Post procedure imaging would verify destruction of targeted cells.

Nanoparticles can be used in a therapeutic modality. There are two types of nanoparticle-based therapeutic formulations: (1) those where the therapeutic molecules are the nanoparticles (therapeutic functions as its own carrier); and (2) those where the therapeutic molecules are directly coupled (functionalized, entrapped or coated) to a carrier. The former category has been traditionally viewed as nanosized therapeutic compounds while the latter are compositions in which the therapeutic compound is bound by or on nanoparticle composed of a different material.

The present invention comprises magnetic nanoparticles wherein the nanoparticles are functionalized with polyvalent ligands capable of binding the same target cells and wherein, upon subjection to a magnetic field, the nanoparticle vortices shift, creating an oscillation which transmits a mechanical force to the target cells thereby inducing apoptosis. The ligands in the present invention may be antibodies, nucleic acid apatamers that polyvalently bind to cancer markers, bacterial antigens, viral antigens, arterial plaque antigens, or fat cell markers.

The present invention comprises a method of diagnosing the presence of a cell population in a mammal comprising administering to said mammal a magnetic nanoparticle composition wherein the nanoparticles of said composition are functionalized with polyvalent ligands capable of binding to target cells of said cell population and subjecting mammal to magnetic field and imaging said nanoparticles.

The present invention comprises a method of treating a targeted tissue or cell comprising administering to said targeted tissue or cell an effective amount of a pharmaceutical composition comprising magnetic nanoparticles functionalized with polyvalent ligands capable of binding a target tissue or cell and subjecting the targeted tissue or cells to a magnetic field thereby inducing a shift in the nanoparticles vortices, wherein the shift transmits a mechanical force to the targeted cells and induce apoptosis.

1. A method of identifying and killing a target cell in a mammal comprising the steps of
a. administering to the mammal a composition comprising a magnetic nanoparticle functionalized with ligands that bind the target cell in a polyvalent manner;
b. subjecting the mammal to a magnet capable of shifting the vortices of the magnetic nanoparticles wherein the shift is detected by an imaging device and wherein the imaging enables detection of the location of the target cells;
c. subsequently administering a therapeutic amount of a composition comprising a magnetic nanoparticles functionalized with ligands that bind the target cell; and
d. subjecting the mammal to a magnet capable of shifting the vortices of the magnetic nanoparticles wherein the shift causes a non-heating mechanism of action wherein ion channels of target cells are disrupted, apoptosis is induced, and the targeted cells die.

2. The method of claim 1 wherein the target cell is selected from the group consisting of bacterial pathogen, viral pathogen, fungal cell, cancer cell, and fat cell.

3. The method of claim 1 wherein the ligand is selected from the group consisting of antibody, DNA aptamer, and RNA aptamer.

4. A method of treating a targeted tissue or cell comprising administering to said targeted tissue or cell an effective amount of a pharmaceutical composition comprising magnetic nanoparticles functionalized with polyvalent ligands capable of binding a target tissue or cell and subjecting the targeted tissue or cell to a magnetic field thereby inducing a shift in the nanoparticle vortices, wherein the shift transmits a mechanical force to the targeted tissue or cell and induce apoptosis

5. A diagnostic and therapeutic composition comprising magnetic nanoparticles functionalized with polyvalent ligands capable of binding target cells, wherein the nanoparticles comprise vortices and wherein, upon subjection to a magnetic field, the nanoparticle vortices shift and wherein the shift is detected using magnetic resonance imaging techniques.
